(19) United States
(12) Patent Application Publication
(10) Pub. No.: US 2017/0166493 A1
(43) Pub. Date: Jun. 15, 2017

(54) SAPO CATALYST FOR A HIGH PRESSURE MTO PROCESS

(71) Applicant: UOP LLC, Des Plaines, IL (US)

(72) Inventors: Nicholas J. Schoenfeldt, Chicago, IL (US); Jaime G. Moscoso, Mount Prospect, IL (US); Wolfgang A. Spieker, Glenview, IL (US); Thulasidas Chellppannair, Cave Creek, AZ (US)

(21) Appl. No.: 14/966,046

(22) Filed: Dec. 11, 2015

(51) Int. Cl. C07C 1/20; C07C 6/04; C07C 4/02

(52) U.S. Cl. CPC C07C 1/20; C07C 4/02; C07C 6/04; C07C 2529/85

(57) ABSTRACT

A process is presented for the conversion of oxygenates to olefins. The process utilizes a catalyst comprising a silicoaluminophosphate with an added basic metal oxide. The basic metal oxide modifies the selectivity to increase the yields of propylene and heavier olefins. The increase in heavier olefins are processed downstream to further increase propylene yields, and to generate a process stream comprising butylenes.

### CROSS REFERENCE TO RELATED APPLICATION

This application claims the benefit of U.S. Provisional Application No. 62/090,948 which was filed on Dec. 12, 2014, the contents of which are hereby incorporated by reference in its entirety.

### FIELD OF THE INVENTION

The present invention relates to the conversion of oxygenates to olefins. In particular, this invention relates to the conversion of methanol to light olefins.

### BACKGROUND

Light olefins serve as feed materials for the production of numerous chemicals. Light olefins have traditionally been produced through the processes of steam or catalytic cracking. The limited availability and high cost of petroleum sources, however, has resulted in a significant increase in the cost of producing light olefins from such petroleum sources.

The search for alternative materials for light olefin production has led to the use of oxygenates such as alcohols and, more particularly, to the use of methanol, ethanol, and higher alcohols or their derivatives. The oxygenates are often produced from more plentiful sources of raw materials, such as conversion of natural gas to alcohols, or the production of oxygenates from coal. Molecular sieves such as microporous crystalline zeolite and non-zeolitic catalysts, particularly silicoaluminophosphates (SAPO), are known to promote the conversion of oxygenates to hydrocarbon mixtures, particularly hydrocarbon mixtures composed largely of light olefins.

The amounts of light olefins resulting from such processing can be further increased by reacting, i.e., cracking, heavier hydrocarbon products, particularly heavier olefins such as C₄ and C₅ olefins, to light olefins. For example, commonly assigned, U.S. Pat. No. 5,914,433 to Marker, the entire disclosure of which is incorporated herein by reference, discloses a process for the production of light olefins comprising olefins having from 2 to 4 carbon atoms per molecule from an oxygenate feedstock. The process comprises passing the oxygenate feedstock to an oxygenate conversion zone containing a metal aluminophosphate catalyst to produce a light olefin stream. A propylene and/or mixed butylene stream is fractionated from said light olefin stream and cracked to enhance the yield of ethylene (C₂H₄) and propylene (C₃H₆) products. This combination of light olefin product and propylene and butylene cracking in a riser cracking zone or a separate cracking zone provides flexibility to the process which overcomes the equilibrium limitations of the aluminophosphate catalyst. In addition, the invention provides the advantage of extended catalyst life and greater catalyst stability in the oxygenate conversion zone.

### SUMMARY

The present invention provides for an improved methanol to olefins (MTO) conversion process.

A first embodiment of the invention is a process for the production of light olefins from an oxygenate feed, comprising passing the oxygenate feed to an MTO reactor, wherein the reactor comprises an MTO catalyst comprising a silicoaluminophosphate, wherein the catalyst selectivity is modified with a basic metal oxide additive, and is operated at reaction conditions to generate an effluent stream comprising olefins. An embodiment of the invention is one, any or all of prior embodiments in this paragraph up through the first embodiment in this paragraph wherein the MTO reactor is a fluidized bed, fixed bed or swing fixed bed. An embodiment of the invention is one, any or all of prior embodiments in this paragraph up through the first embodiment in this paragraph wherein the metal in the basic metal oxide additive is selected from the group consisting of metals in groups 1-4 of the periodic table as well as the lanthanides and actinides. An embodiment of the invention is one, any or all of prior embodiments in this paragraph up through the first embodiment in this paragraph wherein the metal in the metal oxide is selected from the group consisting of sodium, scandium, yttrium, lanthanum, cerium, actinium, calcium, magnesium, and mixtures thereof. An embodiment of the invention is one, any or all of prior embodiments in this paragraph up through the first embodiment in this paragraph wherein the silicoaluminophosphate is SAPO-18, SAPO-34, SAPO-5 or combinations thereof. An embodiment of the invention is one, any or all of prior embodiments in this paragraph up through the first embodiment in this paragraph wherein the metal oxide is yttrium oxide. An embodiment of the invention is one, any or all of prior embodiments in this paragraph up through the first embodiment in this paragraph wherein the oxygenates comprise alcohols, aldehydes and ethers. An embodiment of the invention is one, any or all of prior embodiments in this paragraph up through the first embodiment in this paragraph wherein the oxygenate comprises methanol and dimethyl ether. An embodiment of the invention is one, any or all of prior embodiments in this paragraph up through the first embodiment in this paragraph wherein the oxygenate comprises methanol. An embodiment of the invention is one, any or all of prior embodiments in this paragraph up through the first embodiment in this paragraph wherein the process pressure and temperature are adjusted to provide a desired butylenes to ethylene ratio. An embodiment of the invention is one, any or all of prior embodiments in this paragraph up through the first embodiment in this paragraph wherein the inlet partial pressure of the oxygenate is between 0.1 and 2.5 MPa. An embodiment of the invention is one, any or all of prior embodiments in this paragraph up through the first embodiment in this paragraph wherein the process temperature is between 300 and 500° C. An embodiment of the invention is one, any or all of prior embodiments in this paragraph up through the first embodiment in this paragraph further comprising passing the effluent stream to a light olefins recovery unit. An embodiment of the invention is one, any or all of prior embodiments in this paragraph up through the first embodiment in this paragraph further comprising passing the effluent stream to a quench tower to generate a water stream and a dewatered effluent stream; passing the dewatered effluent stream to a compressor to generate a compressed stream; passing the compressed stream to a DME recovery unit to generate a DME stream and a DME olefins stream; and passing the DME olefins stream to a light olefins recovery unit to generate an ethylene stream, a propylene stream and a C₄⁺ heavies stream. An embodiment of the invention is one, any or all of prior embodiments in this paragraph up through the first embodiment in this paragraph further comprising passing the heavies stream to an olefin cracking unit to generate an olefins cracking effluent stream comprising light olefins. An embodiment of the invention is one, any or all of prior embodiments in this paragraph up through the first embodiment in this paragraph further comprising passing the olefins cracking effluent stream to the light olefins recovery unit. An embodiment of the invention is one, any or all of prior embodiments in this paragraph up through the first embodiment in this paragraph further comprising passing the effluent stream to a quench tower to generate a water stream and a dewatered effluent stream; passing the dewatered effluent stream to a compressor to generate a compressed stream; passing the compressed stream to a DME recovery unit to generate a DME stream and a DME olefins stream; and passing the DME olefins stream to a light olefins recovery unit to generate an ethylene stream, a propylene stream, a C₄ olefin stream and a C₅⁺ heavies stream. An embodiment of the invention is one, any or all of prior embodiments in this paragraph up through the first embodiment in this paragraph further comprising passing a portion or all of the C₄ olefin stream and a portion or all of the ethylene stream to a metathesis reactor, thereby generating a metathesis stream comprising propylene. An embodiment of the invention is one, any or all of prior embodiments in this paragraph up through the first embodiment in this paragraph further comprising passing the C₅⁺ heavies stream to an olefin cracking unit to generate an olefins cracking effluent stream comprising light olefins. An embodiment of the invention is one, any or all of prior embodiments in this paragraph up through the first embodiment in this paragraph further comprising passing the olefins cracking effluent stream to the light olefins recovery unit. An embodiment of the invention is one, any or all of prior embodiments in this paragraph up through the first embodiment in this paragraph further comprising passing the effluent stream to a quench tower to generate a water stream and a dewatered effluent stream; passing the dewatered effluent stream to a compressor to generate a compressed stream; passing the compressed stream to a DME recovery unit to generate a DME stream and a DME olefins stream; and passing the DME olefins stream to a light olefins recovery unit to generate an ethylene stream, a propylene stream, a C₄ olefin stream, a C₅ olefin stream and a C₆⁺ heavies stream. An embodiment of the invention is one, any or all of prior embodiments in this paragraph up through the first embodiment in this paragraph further comprising passing a portion or all of the C₅ stream and a portion or all of the ethylene stream to a metathesis reactor, thereby generating a metathesis stream comprising propylene and butenes. An embodiment of the invention is one, any or all of prior embodiments in this paragraph up through the first embodiment in this paragraph further comprising passing the C₄ stream to a C₄ separation unit to generate an isobutene stream and normal butene stream. An embodiment of the invention is one, any or all of prior embodiments in this paragraph up through the first embodiment in this paragraph further comprising passing the normal butene stream to an oxidative dehydrogenation reactor, thereby generating an on purpose butadiene stream consisting of butadiene. An embodiment of the invention is one, any or all of prior embodiments in this paragraph up through the first embodiment in this paragraph further comprising passing the C₆⁺ heavies stream to an olefin cracking unit to generate an olefins cracking effluent stream comprising light olefins. An embodiment of the invention is one, any or all of prior embodiments in this paragraph up through the first embodiment in this paragraph further comprising passing the olefins cracking effluent stream to the light olefins recovery unit. An embodiment of the invention is one, any or all of prior embodiments in this paragraph up through the first embodiment in this paragraph further comprising passing the effluent stream to a quench tower to generate a water stream and a dewatered effluent stream; passing the dewatered effluent stream to a compressor to generate a compressed stream; passing the compressed stream to a DME recovery unit to generate a DME stream and a DME olefins stream; and passing the DME olefins stream to a light olefins recovery unit to generate an ethylene stream, a propylene stream, a C₄ olefin stream, a C₅ product stream and a C₆⁺ heavies stream. An embodiment of the invention is one, any or all of prior embodiments in this paragraph up through the first embodiment in this paragraph further comprising passing a portion or all of the C₅ heavy stream and a portion or all of the ethylene product stream to a metathesis reactor, thereby generating a metathesis stream comprising propylene and butenes. An embodiment of the invention is one, any or all of prior embodiments in this paragraph up through the first embodiment in this paragraph further comprising passing the C₄ product stream to a C₄ separation unit to generate an isobutene stream and normal butene stream. An embodiment of the invention is one, any or all of prior embodiments in this paragraph up through the first embodiment in this paragraph further comprising passing the C₆⁺ heavies stream to an olefin cracking unit to generate an olefins cracking effluent stream comprising light olefins. An embodiment of the invention is one, any or all of prior embodiments in this paragraph up through the first embodiment in this paragraph further comprising passing the olefins cracking effluent stream to the light olefins recovery unit. An embodiment of the invention is one, any or all of prior embodiments in this paragraph up through the first embodiment in this paragraph wherein the inlet partial pressure of the oxygenate is greater than 100 kPa. An embodiment of the invention is one, any or all of prior embodiments in this paragraph up through the first embodiment in this paragraph wherein the inlet partial pressure of the oxygenate is greater than 200 kPa. An embodiment of the invention is one, any or all of prior embodiments in this paragraph up through the first embodiment in this paragraph wherein the inlet partial pressure of the oxygenate is greater than 300 kPa.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

### DETAILED DESCRIPTION

The production of light olefins, ethylene and propylene, are important precursors for products today, Most notably, the principal products are polyethylene and polypropylene. The source of these precursors has been mainly from the cracking of naphtha. Increasingly, other sources for the production of light olefins is sought due to cost considerations and availability of raw materials. Oxygenate, notably methanol, can be converted and is increasingly being used. Methanol can be generated from several sources, including natural gas and coal.

The methanol to olefin (MTO) process has been successfully commercialized. U.S. Pat. No. 6,303,839 presents an integrated MTO-olefin cracking process. The oxygenate feedstock is catalytically converted over a silicoaluminophosphate (SAPO) catalyst. The increase in light olefin production is also described in U.S. Pat. No. 7,317,133 wherein the production of heavier olefins are directed to an olefin cracking reactor to generate a process stream comprising light olefins. The olefin cracking process utilizes a different catalyst from a family of crystalline silicate having an MFI or MEL. Examples of these catalysts include ZSM-5 or ZSM-11.

Additional process developments continue to be generated, such as U.S. Pat. No. 7,568,016 that integrates the MTO with an ethylene dimerization process and metathesis process for increasing the propylene yields. The dimerization process can also be used to increase the heavier olefins for other purposes. U.S. Pat. No. 7,732,650 describes a process for the separation of butenes, along with isomerization and metathesis reactions.

Additional process developments continue to be generated, such as U.S. Pat. No. 7,568,016 that integrates the MTO with an ethylene dimerization process and metathesis process for increasing the propylene yields. The dimerization process can also be used to increase the heavier olefins for other purposes. U.S. Pat. No. 7,732,650 describes a process for the separation of butenes, along with isomerization and metathesis reactions.

Other aspects include controlling the process with modifications of the catalyst, such as limiting the Si/Al2 ratio to between 0.10 and 0.32 as in U.S. Pat. No. 7,763,765.

The MTO process using a SAPO catalyst is believed to rely on occluded coke for the production of light olefins. This occluded coke is required as co-catalyst allowing the olefin formation and contributes to the selectivation of the catalyst toward the lightest products, as shape selectivity within the catalyst pore structure improves with the increase in coke size. Due to its co-catalytic role inside the catalyst micropores, coke is a necessary by-product of the MTO reaction. However, the increase in coke also is associated with the deactivation of the catalyst, and speeds the deactivation with the coke growth. Therefore, methods or catalysts found to allow improved product yield with reduced occluded coke yield provide an increase in process efficiency and therefore an increase in profit for light olefin producers.

Addition of basic metal oxides to typical MTO catalysts results in improved (reduced) coke yields and deactivation rates. Additionally, for SAPO-34 and SAPO-18, along with the reduction of coke selectivity there is a reduction in the ethylene production. This is presumably due to the reduced size/shape selectivity afforded by the coke that is formed when a basic metal oxide is present. To balance this there is an increase in heavier olefins, and in particular butenes. For a process where maximized ethylene and propylene yields are desired, this can lead to an overall increase with downstream processing of the butylenes to increase light olefin production, and especially propylene production. However, if one desires an olefin stream with added propylene, butylenes and/or other heavy olefins, this can lead to a product with a more desired olefin distribution.

The present invention provides for an improvement in the propylene and butylene yields from an MTO process. The addition of a basic metal oxide to SAPO catalysts results in reduced coke yields and slower deactivation rates, which concurrently improves propylene, butylenes and heavier olefin yields. Furthermore, the present invention discloses methods for altering the MTO process conditions to further maximize the propylene, butylenes and heavier olefin yields over the improved basic metal oxide-containing and SAPO-containing catalyst.

The process includes passing an oxygenate feed to an MTO reactor. The reactor includes a catalyst comprising a silicoaluminophosphate, or SAPO, wherein the catalyst is modified with a basic metal oxide additive. The reactor is operated at reaction conditions to generate an effluent stream comprising olefins. The reactor can be a fluidized bed reactor, a fixed bed reactor or a swing fixed bed reactor. Preferred SAPOs include SAPO-18, SAPO-34, and SAPO-5. Combinations of SAPO catalysts can also be used in the MTO reactor.

The oxygenates can comprise alcohols, aldehydes, and ethers. Preferred oxygenates include methanol and dimethyl ether.

The basic metal oxide additive includes a metal selected from the group consisting of metals in groups 1-4 of the periodic table, lanthanides and actinides. Preferred metals in the metal oxide include sodium, scandium, yttrium, lanthanum, cerium, actinium, calcium, magnesium, and mixtures thereof. A most preferred metal oxide is yttrium oxide, or yttria.

The process is operated to obtain a desired butylenes to ethylene ratio. The butylenes and ethylene can be passed to a metathesis reactor to increase the propylene production. The theoretical ratio is 2:1, but the reaction to maximize propylene production from metathesis requires a ratio greater than 2:1.

The reaction conditions for the MTO process include a pressure between 100 kPa and 2.5 MPa (absolute) in the reactor, with a temperature between 300° C. and 500° C. A preferred temperature is between 350 C and 500 C. Preferred pressures include oxygenate partial pressures at the inlet of the reactor to be greater than 100 kPa, with a more preferred oxygenate partial pressure greater than 200 kPa. Preferred pressures for the reactor includes pressure between 200 kPa and 2 MPa (absolute).

The process further can includes passing the effluent stream to a light olefins recovery unit. The effluent stream is passed to a quench tower to generate a water stream and a dewatered effluent stream. The dewatered effluent stream is passed to a compressor to generate a compressed stream, and the compressed stream is passed to a DME recovery unit. The DME recovery unit generates a DME stream, comprising dimethyl ether, and a DME olefins stream comprising light olefins. The DME olefin stream is passed to the light olefins recovery unit and generates an ethylene stream, a propylene stream, a C4 stream and a C5+ heavies stream. In a variation, the C4 stream and C5+ stream can be a combined process stream from the light olefins recovery unit.

In one embodiment, the heavies stream can be passed to an olefin cracking unit to generate an olefins cracking effluent stream comprising light olefins. The heavies stream can comprise the C5+ heavies stream, or can be a combination of the C4 stream and C5+ stream. The olefins cracking effluent stream is passed to the light olefins recovery unit to further recovery ethylene and propylene.

In another embodiment, the process further includes passing a portion or all of the C4 olefin stream and a portion or all of the ethylene stream to a metathesis reactor to generate a metathesis stream comprising mostly propylene. The metathesis stream is passed to the light olefins recovery unit to recover the propylene.

In another embodiment, the process further includes passing a portion or all of the C5+ stream and a portion or all of the ethylene stream to a metathesis reactor. The metathesis reactor with the heavier olefins will generate a metathesis stream comprising propylene and butenes. The metathesis stream is passed to a separation unit to generate a C4 stream and a propylene stream. The C4 stream is sent to a C4 separation unit to generate an isobutene stream and a normal butene stream. The process can further include passing the normal butene stream to an oxidative dehydrogenation reactor to generate an on-purpose butadiene stream comprising 1,3 butadiene.

It has been discovered that the addition of the metal oxide to the SAPO catalyst shifts the olefin product distribution from the MTO reactor to generate an increased heavier olefin distribution. This increase in heavier olefins allows for additional ease in downstream processing to increase propylene production, and for the production of butadienes. Existing methods, known to those skilled in the art, for adding basic metal oxides to SAPO catalysts can be used.

The results of several tests show the change in the MTO product stream composition in Table 1. As can be seen in the results, the addition of the metal oxide has increased the proportion of heavies relative to the catalyst without the metal oxide, and has also increase the butylenes to ethylene ratios.

TABLE 1
MTO reactor product compositions

| | C2═ % | C3═ % | C4═ % | C4═/C2═ |
|---|---|---|---|---|
| Catalyst | (C %) | (C %) | (C %) | (C %) |
| SAPO-34 | 14.3 | 42.1 | 26.1 | 1.8 |
| SAPO-34 + Y2O3 | 12.2 | 39.5 | 27.6 | 2.3 |
| SAPO-18 | 11.7 | 38.6 | 31.8 | 2.7 |
| SAPO-18 + Y2O3 | 10.9 | 38.6 | 33.2 | 3.1 |

While the invention has been described with what are presently considered the preferred embodiments, it is to be understood that the invention is not limited to the disclosed embodiments, but it is intended to cover various modifications and equivalent arrangements included within the scope of the appended claims.

1. A process for the production of light olefins from an oxygenate feed, comprising:
passing the oxygenate feed to an oxygenate to olefins reactor, wherein the reactor comprises an catalyst comprising a silicoaluminophosphate, wherein the catalyst selectivity is modified with a basic metal oxide additive, and is operated at reaction conditions to generate an effluent stream comprising light olefins with a butylene to ethylene ratio between about 1.8 and about 3.1; and
passing a portion of the effluent stream comprising light olefins to a metathesis reactor to generate a metathesis stream comprising propylene.

2. The process of claim 1 wherein the oxygenate to olefins reactor is a fluidized bed, fixed bed or swing fixed bed.

3. The process of claim 1 wherein the metal in the basic metal oxide additive is selected from the group consisting of metals in groups 1-4 of the periodic table as well as lanthanides and actinides.

4. The process of claim 3 wherein the metal in the basic metal oxide is selected from the group consisting of sodium, scandium, yttrium, lanthanum, cerium, actinium, calcium, magnesium, and mixtures thereof.

5. The process of claim 1 wherein the silicoaluminophosphate is SAPO-18, SAPO-34, SAPO-5 or combinations thereof.

6. The process of claim 1 wherein the oxygenates comprise alcohols, aldehydes and ethers.

7. The process of claim 1 wherein a process pressure and temperature are adjusted to provide a desired butylenes to ethylene ratio.

8. The process of claim 1 wherein an inlet partial pressure of the oxygenate is between 0.1 and 2.5 MPa.

9. The process of claim 1 wherein a process temperature is between 300 and 500° C.

10. The process of claim 1 further comprising passing the effluent stream to a light olefins recovery unit.

11. The process of claim 1 further comprising:
passing the effluent stream to a quench tower to generate a water stream and a dewatered effluent stream;
passing the dewatered effluent stream to a compressor to generate a compressed stream;
passing the compressed stream to a DME recovery unit to generate a DME stream and a DME olefins stream; and
passing the DME olefins stream to a light olefins recovery unit to generate an ethylene stream, a propylene stream and a C₄⁺ heavies stream; wherein at least the C₄₊ heavies stream is the portion of the effluent stream that is passed to a metathesis reactor.

12. (canceled)

13. The process of claim 11 further comprising passing the olefins cracking effluent stream to the light olefins recovery unit.

14. The process of claim 1 further comprising:
passing the effluent stream to a quench tower to generate a water stream and a dewatered effluent stream;
passing the dewatered effluent stream to a compressor to generate a compressed stream;
passing the compressed stream to a DME recovery unit to generate a DME stream and a DME olefins stream; and
passing the DME olefins stream to a light olefins recovery unit to generate an ethylene stream, a propylene stream, a C₄ olefin stream and a C₅⁺ heavies stream; wherein at least the C₄ olefins stream is the portion of the effluent stream that is passed to a metathesis reactor.

15. The process of claim 14 wherein the portion of the effluent stream that is passed to a metathesis reactor further comprises a portion of the ethylene stream.

16. The process of claim 14 further comprising passing the C₅⁺ heavies stream to an olefin cracking unit to generate an olefins cracking effluent stream comprising light olefins.

17. The process of claim 16 further comprising passing the olefins cracking effluent stream to the light olefins recovery unit.

18. The process of claim 14 further comprising passing the C₄ olefin stream to a C₄ separation unit to generate an isobutene stream and normal butene stream.

19. The process of claim 1 wherein an inlet partial pressure of the oxygenate is greater than 200 kPa.

20. The process of claim 19 wherein an inlet partial pressure of the oxygenate is greater than 300 kPa.
